(19) United States
(12) Patent Application Publication
(10) Pub. No.: US 2007/0141167 A1
(43) Pub. Date: Jun. 21, 2007

(54) Use of Benzyl Nicotinate for Pain Relief

(75) Inventors: Anthony Tesoriero JR., Elmont, NY (US); Irwin Palefsky, Weehawken, NJ (US); Ni'Kita Wilson, Union, NJ (US)

Correspondence Address: CHERYL H AGRIS PHD, PO BOX 806, PELHAM, NY, 10803, US

(73) Assignee: Doctor Bronze Solar Potions, Inc, Elmont, NY (US)

(21) Appl. No.: 11/548,176

(22) Filed: Oct. 10, 2006

Related U.S. Application Data
(60) Provisional application No. 60/725,992, filed on Oct. 11, 2005.

(51) Int. Cl. A61K 35/12; A61K 36/61; A61K 36/534; A61K 31/455; A61K 31/7008; A61K 31/737

(52) U.S. Cl. 424/522; 424/742; 424/771; 514/355; 514/54; 514/62; 424/747

(57) ABSTRACT

The invention is directed to novel topical compositions for relieving pain comprising benzyl nicotinate as well as methods of using these compositions.

### PRIORITY CLAIM

This application claims priority to application no. 60/725,992, filed Oct. 11, 2005, the contents of which are incorporated herein by reference.

### FIELD OF THE INVENTION

The invention is directed to novel compositions for relieving pain involving the use of benzyl nicotinate.

### BACKGROUND OF THE INVENTION

Pain Relief

There has recently been a great deal of interest in pain relief, particularly in those suffering from chronic pain. Patients respond much better to treatment for a wide variety of serious physical and neurological injuries and disorders if the pain associated with those conditions can be relieved. Further, it is desirable if there are preparations that may be self administered and/or may be administered topically without side effects.

Benzyl Nicotinate

A number of uses have been disclosed for benzyl nicotinate. Examples are disclosed below.

U.S. Pat. No. 4,455,146, discloses a plaster bandage containing a medicinal component. One of the possible medicinal components mentioned is benzyl nicotinate.

U.S. Pat. No. 4,713,397, discloses and claims the use of a ubiquinone for reducing fall-out of hair. It also discloses a composition comprising ubiquinone and a vasodilator (carpronium chloride, vitamin E nicotinate and benzyl nicotinate). In one composition, benzyl nicotinate is present in 2% by weight.

U.S. Pat. No. 4,849,418, discloses and claims analgesic, anti-inflammatory, antirheumatic “transdermally absorbable water-containing preparations of therapeutically active arylpropionic acid derivatives”. It is also stated that these compositions may further comprise up to 2% by weight a “blood circulation-promoting or rubefacient substance selected from the group consisting of benzyl nicotinate and a salicylate”.

U.S. Pat. No. 5,658,576, discloses and claims a method for treating cellulitis or reducing localized fatty excesses by applying a composition in the form of an oil, balm, oil in water or water in oil emulsion, containing 0.1-10% by weight alpha-tocopherol nicotinate, benzyl nicotinate, xanthinol nicotinate, hexyl nicotinate, and/or alpha-tocopherol acetate which is free of any other lipolytic substance having phophodiesterase inhibiting activity or oil-soluble plant extract having lipolytic or body slimming activity.

U.S. Pat. No. 6,149,468, discloses a hair stimulant composition comprising p-methane-3,8-diol and a blood circulation promoter (e.g., capromium chloride, vitamin E derivative, cepharanthin, benzyl nicotinate, capsicum tincture, Swertiae herba extract or Spilanthes acmella extract), 5-alpha-reductase inhibitor, an antihistamine, cell activator, antiphlogistic and/or antimicrobial. Benzyl nicotinate is present in 0.05% by weight.

U.S. Pat. No. 6,479,059, discloses a cosmetic composition for reactivating hair growth comprising cysteine, lysine or derivatives thereof, glycoprotein and optionally a nicotinic ester (e.g., benzyl nicotinate-0.05-5% by weight). US Patent Appln. Publication No. 20060057093 also discloses a composition for combating hair loss comprising benzyl nicotinate, hydroxyproline and aspartic acid, complexed with a silanol, —an enzyme activator, comprising octyl butyrate is described.

U.S. Patent Appln. Publication No. 2004/0254202, discloses and claims a method for promoting sleep by topically applying a non-hypnotic vasoactive agent (e.g., nicotinates), which may be present in the amount of 0.001-25% by weight.

U.S. Patent Appln. Publication No. 2005/0048008, discloses an antiaging “cosmetic delivery system” comprising (i) an extracellular antioxidant or free-radical neutralizing composition; (ii) an intracellular antioxidant or free-radical neutralizing composition; (iii) an anti-inflammatory composition; (iv) collagen or fibrin boosting composition and (v) carrier base or topical delivery system. One of the anti-inflammatories recited was benzyl nicotinate (0.0001%-10% by weight).

U.S. Patent Appln. Publication No. 2005/0058672, discloses and claims compositions that may be used to treat diaper rash. The composition contains: (i) at least one divalent metal cation and zeolite anion ion-pair and/or or a quaternary ammonium cation and zeolite anion ion-pair composition, (ii) at least one anti-inflammatory composition, (iii) at least one water retentive composition, (iv) a cosmetically or pharmaceutically acceptable delivery system or a carrier base composition. Claim 8 recites a number of anti-inflammatory compounds; one of the compounds recited was benzyl nicotinate.

Krzic et al., 2001, J. Controlled Release 70:203-211 discloses the use of benzyl nicotinate as a rubefacient/blood circulation promoter.

Kristl et al., 2003, AAPS PharmSci 5:1-8 discloses the use of benzyl nicotinate for enhancing the effect of radiation on tumors.

### SUMMARY OF THE INVENTION

It has unexpectedly and surprisingly been found that benzyl nicotinate can relieve pain.

Thus, the invention is directed a novel topical composition comprising benzyl nicotinate, which is present preferably in an mount for relieving pain, as well as a method for relieving pain in a subject in need thereof comprising topically applying benzyl nicotinate or composition comprising benzyl nicotinate to said subject in need thereof in an amount effective to relieve pain in said subject. In a particular embodiment, the benzyl nicotinate is present in the composition in an amount effective to relieve pain in a subject in need thereof. The composition may actually be a topical composition for relieving pain comprising benzyl nicotinate, optionally present in the form of an emulsion.

In a more specific embodiment, the composition comprises benzyl nicotinate, eucalyptus, peppermint and menthol. In yet a more specific embodiment, the composition may further comprise volcanic material, glucosamine, chondroitin and/or emu oil.

In yet a more specific embodiment, the composition comprises benzyl nicotinate, eucalyptus oil, peppermint oil, menthol, glucosamine, chondroitin, emu oil, spearmint oil, a second pain relief agent and/or volcanic material

The invention is also directed to use of benzyl nicotinate for the manufacture of a medicament for relieving pain in a subject.

### DETAILED DESCRIPTION OF THE INVENTION

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

As noted above, the compositions of the present invention are topical compositions and in a particular embodiment topical cosmetic skin care compositions. As defined herein, “cosmetic compositions” are intended to be placed in contact with the external parts of a subject (may be a human subject) and act to clean, change appearance and/or keeping the body part to which the composition is applied in good condition. The compositions of the present invention may be used to alleviate pain in a subject. The subject may be a mammal and preferably a human subject. The compositions may be used to alleviate pain resulting from arthritis and relieves minor muscle pain, stiffness, joint pain and athletic soreness.

The compositions of the present invention comprise benzyl nicotinate. In a particular embodiment, such compositions may comprise between about 0.0001 to about 10% benzyl nicotinate by weight. In a particular embodiment, the composition may comprise between about 0.001 to about 2% benzyl nicotinate by weight and preferably between about 0.05-1.25% by weight. The composition may comprise further analgesic agents such as Willow Bark extract. (0.05-3.0) and (0.1-1.5%)[How much? What sort of range should be used?]

The compositions may further comprise glucosamine, chondroitin, eucalyptus oil, menthol, emu oil, peppermint oil, spearmint oil, and/or volcanic material (e.g. zeolite)). In a specific embodiment, the composition comprises benzyl nicotinate and menthol. Menthol may be present in an amount between about 0.01 to about 1% by weight. In yet another embodiment, the composition may also comprise an emollient (e.g., emollient oils such as mineral oil, natural oils and cosmetic emollient esters, particularly, silicone oils such as dimethicone). The emollient may be present an amount of about 25 to about 90% by weight.

In a preferred embodiment, the composition comprises benzyl nicotinate and optionally another analgesic such as willow bark extract, menthol, eucalyptus, preferably eucalyptus oil and peppermint, preferably peppermint oil. Benzyl nicotinate may be present in the amount of about 0.0001 to about 10% benzyl nicotinate by weight, particularly between about 0.001 to about 2% benzyl nicotinate by weight and most preferably between about 0.05-1.25% by weight, menthol may be present in the amount of between about 0.01 to about 10%, eucalyptus oil and peppermint oil may be present in the amount of about 0.05-10% and and preferably between about 0.1 to about 5% by weight. The composition may also further comprise glucosamine, chondroitin and/or emu oil. Glucosamine and chondroitin may each be present in an amount of about 0.001 to about 0.5% by weight, and preferably in the amount of about 0.01 to about 0.5% by weight. Emu oil may be present in the amount of between about 0.1 to about 5.0% by weight and preferably between about 0.25% to about 3.0%. As above, the composition may further comprise an emollient and/or volcanic material.

The compositions of the present invention may be in the form of lotions, creams, gels, sticks, sprays, mousses, viscous liquids, ointments and pastes. These product types may comprise several types of formulations including, but not limited to emulsions, and solids. These formulations preferably contain a “cosmetically acceptable carrier” or “pharmaceutically acceptable carrier” which includes any and all solvents, dispersion media, coatings, antibacterial and antifungal agents, isotonic and absorption delaying agents, surfactants (e.g., lecithin) and the like. Examples of such a dispersing agent include but is not limited to magnesium aluminum silicate, Bentone Gels, cellulosic gums, beeswax (e.g., octyldodecanol beeswax). The dispersing agent generally comprises from about 0.1% to about 5.0%, preferably 0.2% to about 3.0%, more preferably about 0.5% to about 2.0% by weight of the composition of the present invention.

The compositions of the present invention may be applied to the skin in the amount of about 0.1-10 mg/cm² of skin. The composition of the present invention would be applied as needed and may be applied throughout the day.

### EXAMPLE

An example of a composition encompassed by the invention is shown below.

| No. | Phase | Compound | % by weight |
|---|---|---|---|
| 1 | A | Isododecan dimethicone polysilicone-11 | 75.13 |
| 2 | B | Benzyl nicotinate | 1.50 |
| 3 | B | Cyclopentasiloxane cyclohexasiloxane | 5.00 |
| 4 | B | Eucalyptus globulus leaf oil | 0.50 |
| 5 | B | Dromiceius (Emu oil) | 1.00 |
| 6 | B | Peppermint oil | 1.00 |
| 7 | B | Rosemary leaf extract | 0.25 |
| 8 | B | Willow bark extract | 0.25 |
| 9 | C | Menthol | 0.25 |
| 10 | C | Gransheat | 0.10 |
| 11 | C | PEG-12 Dimethicone | 5.00 |
| 12 | D | Zeolite | 10.00 |
| 13 | E | Glucosamine sulfate (1% soln) | 0.01 |
| 14 | E | Chondroitin sulfate (1% soln) | 0.01 |

Phase A is combined and mixed until uniform. Phase B ingredients are combined and mixed until uniform. Phase B is then added to phase A. Phase C ingredients are combined and heated slightly to dissolve menthol. Phase C is then added to the batch containing phase A and phase B ingredients. Phase D is added to the batch and mixed until uniform Finally, phase E is added to the batch.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties.

What is claimed is:

1. A topical composition comprising benzyl nicotinate in an amount effective to relieve pain in a subject in need thereof and a carrier.

2. The composition according to claim 1, wherein said composition is an emulsion.

3. The composition according to claim 1, wherein said benzyl nicotinate is present in an amount ranging between about 0.0001 to about 10% benzyl nicotinate.

4. The composition according to claim 1, wherein said composition further comprises glucosamine, chondroitin, eucalyptus oil, menthol, emu oil, peppermint oil, spearmint oil, and/or volcanic material.

5. The composition according to claim 1, wherein said composition further comprises an emollient.

6. The composition according to claim 1, wherein said composition further comprises a second pain relieving agent.

7. The composition according to claim 6 wherein said second pain relieving agent is willow bark extract.

8. The composition according to claim 7, wherein said willow bark extract is present in the amount of about

9. The composition according to claim 4, wherein eucalyptus oil is present in an amount of about 0.05 to about 10% by weight, peppermint oil is present in the amount of about 0.05 to about 10% by weight, spearmint oil is present in the amount of about 0.05-10%, glucosamine is present in an amount of about 0.001 to about 0.5% by weight, chondroitin is present in an amount of about 0.001 to about 0.5% by weight, Emu oil is present in the amount of between about 0.1 to about 5.0% by weight and zeolite is present in the amount of about 0.01 to about 1% by weight.

10. A method for relieving pain in a subject in need thereof comprising topically applying the composition according to claim 1 to said subject in need thereof in an amount effective to relieve pain in said subject.

11. The method according to claim 10, wherein said composition is applied more than once daily.

12. The method according to claim 10, wherein the subject is a mammal.

13. The method according to claim 10, wherein the subject is a human.

14. The method according to claim 10, wherein the pain is joint pain or muscle pain.

15. A topical composition comprising benzyl nicotinate, eucalyptus oil, peppermint oil and menthol.

16. The composition according to claim 15, wherein said composition is a pain relief composition.

17. A topical composition comprising benzyl nicotinate, eucalyptus oil, peppermint oil, menthol, glucosamine, chondroitin, emu oil, spearmint oil, a second pain relief agent and/or volcanic material.
